(19) United States
(12) Patent Application Publication
(10) Pub. No.: US 2026/0174920 A1
(43) Pub. Date: Jun. 25, 2026

(54) Ultraviolet Sanitation Unit for Plumbing Fixtures

(71) Applicant: Sloan Valve Company, Franklin Park, IL (US)

(72) Inventors: Jacob Suvalskas, Franklin Park, IL (US); Kay Herbert, Winthrop, MA (US); James McSweeney, Franklin Park, IL (US); John Maltby, Chicago, IL (US)

(21) Appl. No.: 19/422,020

(22) Filed: Dec. 16, 2025

Related U.S. Application Data
(63) Continuation of application No. 17/829,420, filed on Jun. 1, 2022, now abandoned.
(60) Provisional application No. 63/195,324, filed on Jun. 1, 2021.

(51) Int. Cl. A61L 2/26; A61L 2/10; E03D 9/00

(52) U.S. Cl. CPC A61L 2/26; A61L 2/10; E03D 9/002

(57) ABSTRACT

A flushometer including a sanitation unit is described herein. The sanitation unit may be configured to emit ultraviolet light in response to the flushometer effectuating a flush of the fixture. The ultraviolet light may sterilize and/or sanitize a plume caused as a result of the flush. The ultraviolet light may be emitted for a predetermined amount of time to sterilize, sanitize, and/or kill any pathogens contained in the plume. Additionally or alternatively, the sanitation unit may emit the ultraviolet light periodically. For example, the sanitation unit may emit the ultraviolet light in response to receiving one or more signals from a gateway and/or server. The sanitation unit may provide an environmentally friendly solution that reduces water consumption and/or cleans, sanitizes, and/or sterilizes plumbing fixtures, toilet seats, etc. between uses without the need for harsh chemicals and/or cleaning solutions.

### CROSS-REFERENCE TO RELATED APPLICATIONS

This application is a continuation of, and claims priority to, U.S. application Ser. No. 17/829,420, filed on Jun. 1, 2022 and entitled “Ultraviolet Sanitation Unit for Plumbing Fixtures,” which is a nonprovisional of, and claims priority to, U.S. Provisional Application No. 63/195,324, filed on Jun. 1, 2021 and entitled “Ultraviolet Sanitation Unit for Plumbing Fixtures,” both of the entireties of which are incorporated herein for all purposes.

### FIELD OF THE DISCLOSURE

Aspects of the disclosure generally relate to a sanitization device and more specifically to an ultraviolet sterilization solution for plumbing fixtures.

### BACKGROUND OF THE DISCLOSURE

Plumbing fixtures may contain pathogens and other harmful material that may be spread through use. For examples, toilets may disperse a plume when flushed. The plume may contain pathogens and other harmful material that may be dispersed throughout a bathroom. The pathogens and other harmful material may come in contact with high-touch surfaces, including, for example, toilet seats. In another example, sink fixtures may contain pathogens and other harmful material through normal handwashing. Thus, there is a need to mitigate the pathogens and other harmful material associated with plumbing fixtures, including those aerosolized and dispersed by flushing a toilet.

### BRIEF SUMMARY OF THE DISCLOSURE

The following presents a simplified summary of various aspects described herein. This summary is not an extensive overview, and is not intended to identify key or critical elements or to delineate the scope of the claims. The following summary merely presents some concepts in a simplified form as an introductory prelude to the more detailed description provided below. Corresponding apparatus, systems, methods, and computer-readable media are also within the scope of the disclosure.

The present disclosure describes an ultraviolet sanitation unit for sterilizing and/or sanitizing plumbing fixtures and, in particular, bathroom fixtures and/or toilets. The ultraviolet sanitation unit may be included in a flushometer, such as a wall-mounted flushometer. The wall-mounted flushometer may comprise a sensor to detect the presence (or absence) of a user. For example, the flushometer may detect when the user has finished and left the range of the sensor. That is, the flushometer may detect when the user has walked away from the plumbing fixture. In another example, a user may clean a fixture and, in response to finishing cleaning the fixture, trigger the sanitation, and/or sterilization cycle. Additionally or alternatively, the user may initiate the sanitation, and/or sterilization cycle, for example, as part of the sanitation, and/or sterilization cycle. The sensor may send a first signal to a solenoid that triggers a flushing action, for example, when the user has finished and left the range of the sensor. Additionally, the sensor may send a second signal to an ultraviolet sanitation unit. The second signal may cause the ultraviolet sanitation unit to emit ultraviolet light, for example, during a flush. The ultraviolet light may be emitted for a predetermined amount of time to neutralize, sterilize, sanitize, and/or kill any pathogens contained in the plume dispersed as a result of the flushing action. Additionally or alternatively, the second signal may be sent to the ultraviolet sanitation unit in response to a user manually activating the flushing action.

In some examples, the flushometer may comprise a transceiver. The transceiver may be configured to receive one or more signals from a gateway. The one or more signals received from the gateway may activate the ultraviolet sanitation unit, for example, as part of a cleaning cycle. That is, the transceiver may receive one or more signals from a server, via the gateway. The transceiver may then send another signal to the ultraviolet sanitation unit. Like above, the signal may cause the ultraviolet sanitation unit to emit ultraviolet light as part of a cleaning/sterilization cycle. The ultraviolet light may be emitted for a predetermined amount of time to disinfect, clean, sanitize, and/or sterilize a plumbing fixture, toilet seat, and/or an area surrounding the pluming fixture (e.g., a water closet).

The ultraviolet sanitizing unit described herein may neutralize, disinfect, clean, sanitize, and/or sterilize a plume dispersed during a flush to reduce the spread of pathogens and other infectious diseases. Moreover, the ultraviolet sanitizing unit described herein may provide an environmentally friendly solution that neutralizes, disinfects, cleans, sanitizes, and/or sterilizes surfaces and/or objects (e.g., plumbing fixtures, plumes dispersed from plumbing fixtures, etc.) periodically and between uses, while reducing water consumption.

The features, along with many others, and benefits are discussed in greater detail below.

### BRIEF DESCRIPTION OF THE DRAWINGS

The present disclosure is described by way of example and not limited in the accompanying figures in which:

FIGS. 1A-1E show an example of a water closet containing a plumbing fixture that includes a sanitation unit;

FIG. 1F shows an in-wall install of a flushometer with a sanitation unit;

FIG. 2 shows an example of an implementation of the hybrid sensor circuit in accordance with one or more aspects of the disclosure;

FIG. 3 shows an example of a sensor according to one or more aspects of the disclosure;

FIGS. 4A and 4B show an example of light array assembly in accordance with one or more aspects of the disclosure;

FIG. 5 shows an example of a process for sanitizing a plumbing fixture according to one or more aspects of the disclosure;

FIG. 6A-6B show an example of sanitizing a plumbing fixture according to one or more aspects of the disclosure;

FIG. 7 shows an example of a process for sanitizing a plurality of plumbing fixtures according to one or more aspects of the disclosure; and

FIGS. 8A and 8B show an example of a process for sanitizing a plurality of plumbing fixtures according to one or more aspects of the disclosure.

### DETAILED DESCRIPTION OF THE DISCLOSURE

In the following description of the various example embodiments, reference is made to the accompanying drawings, which form a part hereof, and in which is shown by way of illustration various example embodiments in which aspects of the disclosure may be practiced. It is to be understood that other embodiments may be utilized and structural and functional modifications may be made without departing from the scope of the present disclosure. Aspects of the disclosure are capable of other embodiments and of being practiced or being carried out in various ways. In addition, it is to be understood that the phraseology and terminology used herein are for the purpose of description and should not be regarded as limiting. Rather, the phrases and terms used herein are to be given their broadest interpretation and meaning.

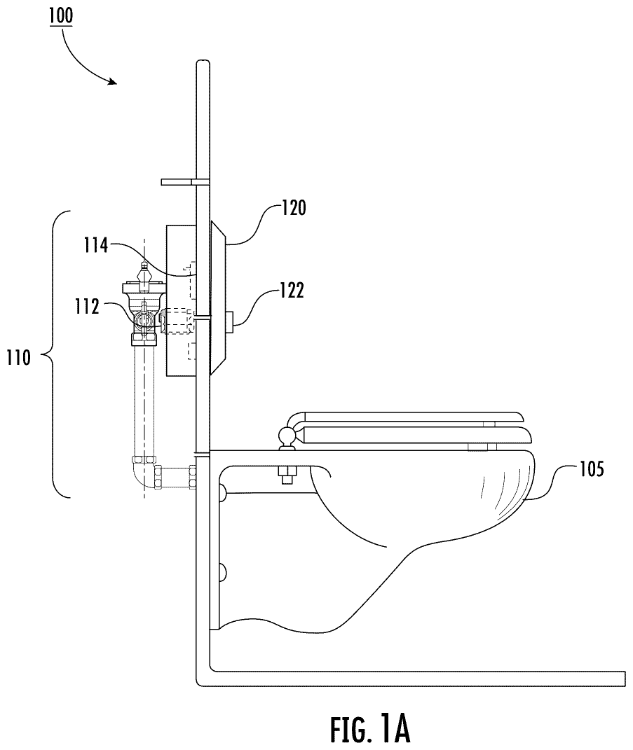

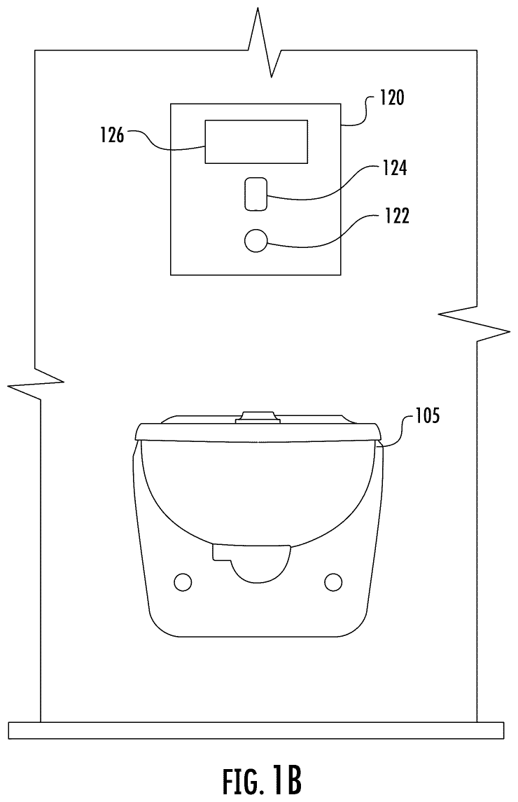

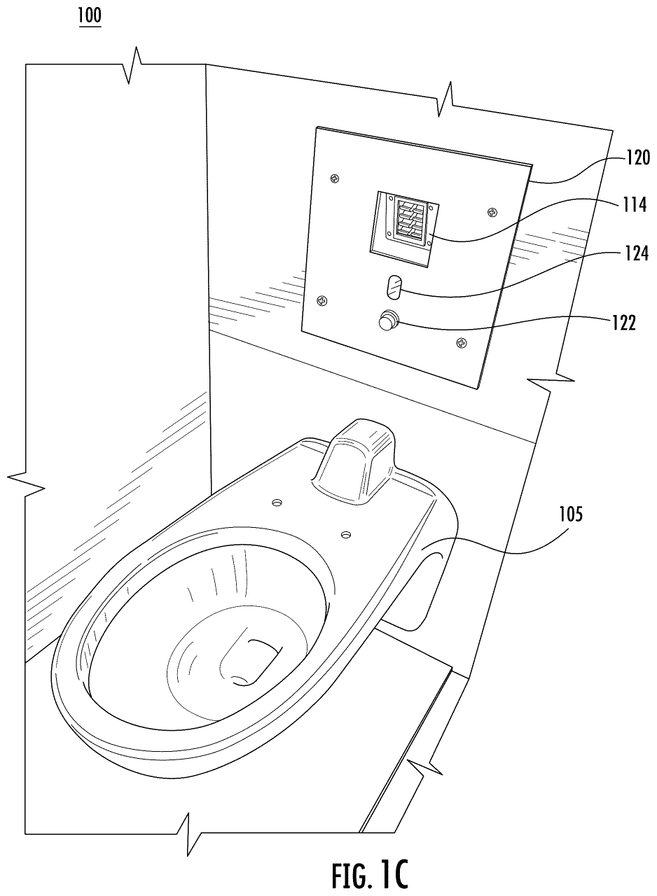

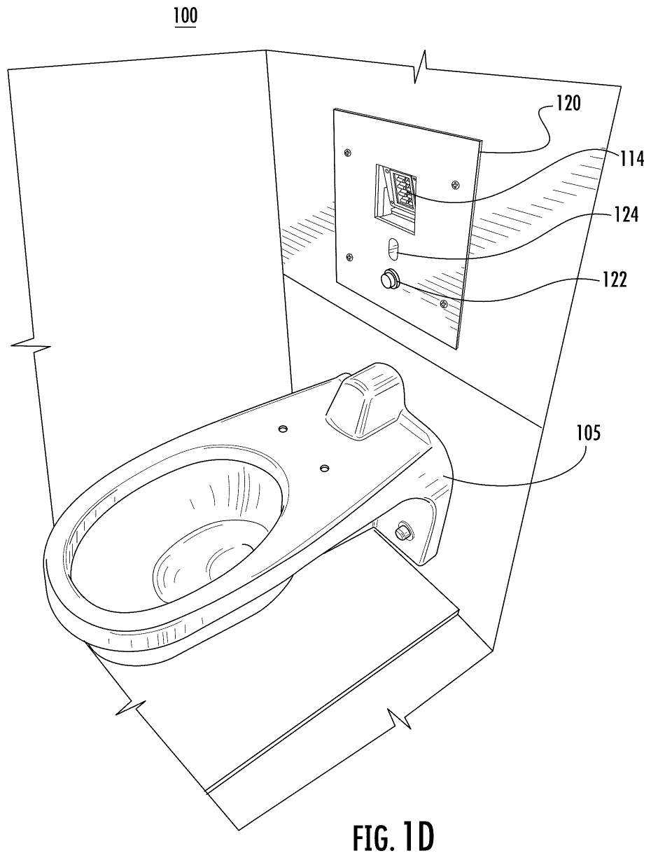

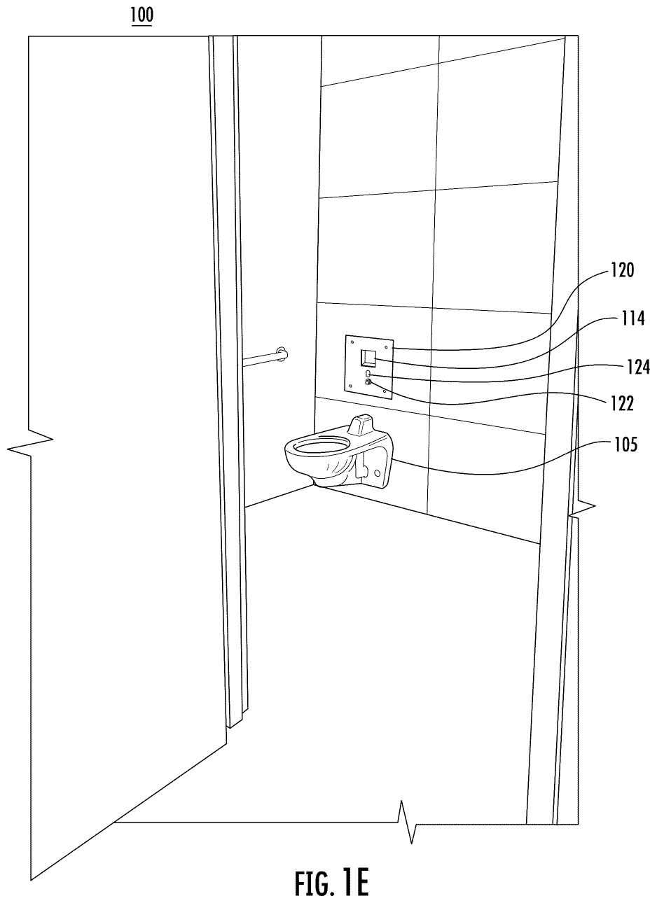

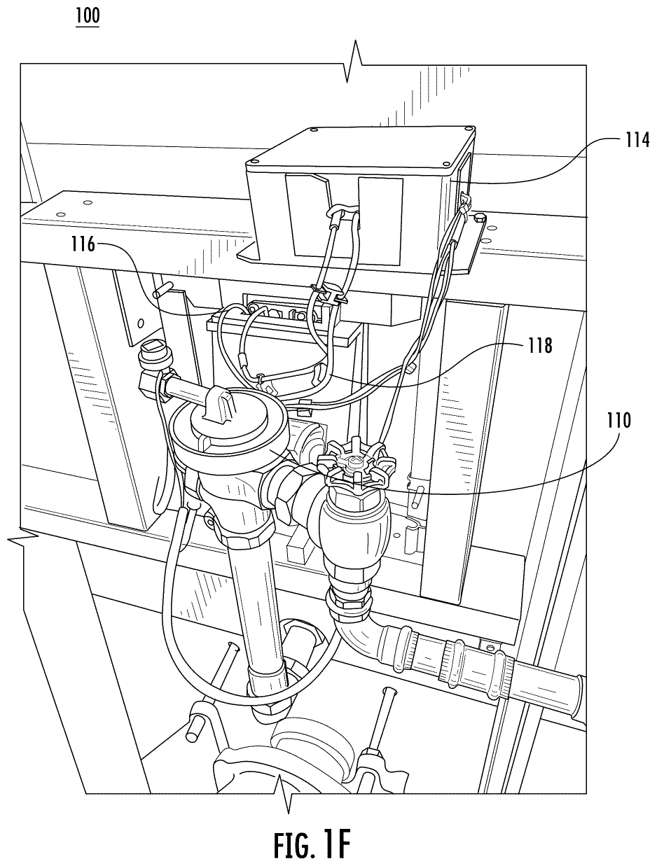

Turning to FIGS. 1A-1E, an example of a water closet 100 is shown. The water closet 100 may comprise a plumbing fixture 105 and a flushometer 110. As shown in FIGS. 1A and 1B, the plumbing fixture 105 may comprise a toilet. However, it will be appreciated that the plumbing fixture 105 may comprise a urinal, a sink, or any other suitable plumbing and/or bathroom fixture.

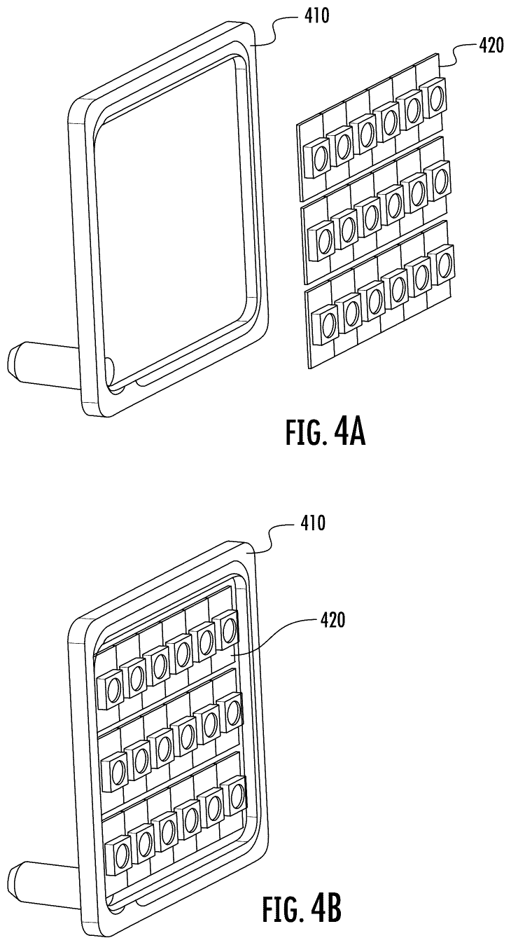

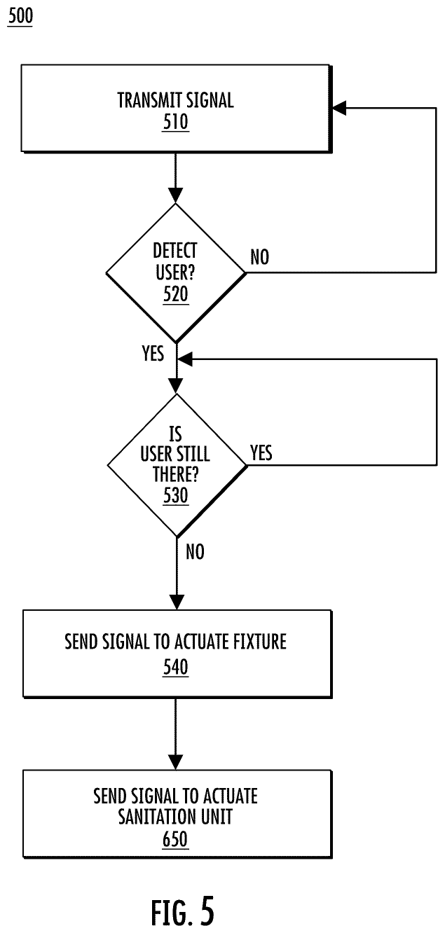

The flushometer may comprise a sensor 112, a sanitation unit 114, a face plate 120, a manual flush button 122, a first cover 124 in the faceplate 120 over the sensor 112, and a second cover 126 in the faceplate 120 over the sanitation unit 114. As will be discussed in greater detail below, the sensor 112 may comprise a time-of-flight (ToF) sensor, an infrared sensor, or a combination thereof. The sensor 112 may be configured to detect the presence (or absence) of a user. Upon detecting a user, the sensor 112 may send a first signal to a solenoid that triggers a flushing action, for example, when the user has finished and/or left the range of the sensor. Additionally, the sensor 112 may send a second signal to the sanitation unit 114. The second signal may cause the sanitation unit 114 to run a cleaning, sanitation, and/or sterilization cycle. The sanitation unit 114 may comprise one or more light arrays. As will be discussed in greater detail with respect to FIG. 4, the one or more light arrays may be configured to emit ultraviolet light of a particular wavelength. In some embodiments, the sanitation unit 114 may be angled downward to neutralize, disinfect, clean, sanitize, and/or sterilize one or more surfaces of the water closet 100 and/or the fixture 105. Additionally or alternatively, the sanitation unit 114 may be configured to neutralize, disinfect, clean, sanitize, and/or sterilize three-dimensional objects, such as fixtures noted above, as well as plumes dispersed from said fixtures. In this way, the sanitation unit 114 may be configured to neutralize, disinfect, clean, sanitize, and/or sterilize two-dimensional and/or three-dimensional surfaces. As part of the cleaning, sanitation, and/or sterilization cycle, the sanitation unit 114 may emit ultraviolet light from one or more light arrays. The ultraviolet light emitted by sanitation unit 114 may be emitted during a flush to sterilize, sanitize, and/or kill any pathogens contained in the plume dispersed as a result of the flushing. The ultraviolet light may be emitted for a predetermined amount of time. By travelling at the speed of light, the ultraviolet light may repeatedly and/or regularly irradiate individual droplets of the plume as thy are dispersed, as well as those that land on any surfaces (e.g., surfaces of a water closet, surfaces of a fixture, the floor, the ceiling, etc.). Additionally or alternatively, the sensor 112, or additional logic, may send a third signal to the sanitation unit 114 to cease emitting the ultraviolet light. In another embodiment, the second signal may be sent to the ultraviolet sanitation unit in response to a user manually activating the flushing action.

As noted above, the faceplate 120 may comprise second cover 126 over the sanitation unit 114. The second cover 126 may comprise a semi-transparent material. In some embodiments, the second cover 126 may comprise a filter, such as an ultraviolet filter. The filter may be configured to allow light, and in particular—ultraviolet light, of a particular wavelength to pass through the second cover 126. In this regard, the filter may be configured to allow UV-C light to pass through the filter from the sanitation unit 114.

FIG. 1F shows that the flushometer may also comprise a power supply 116, wiring to connect to a power supply 118, and/or a communication interface (not shown). The communication interface may be configured to communicate with a gateway and/or server. The communication interface may comprise one or more transceivers, modems, digital signal processors, and/or additional circuitry and software, protocol stack, and/or network stack for communicating via any network, wired or wireless, using any protocol as described herein. The communication interface may comprise a short-range wireless transceiver that provides near field communication (NFC) communications, Bluetooth® communications, Bluetooth® Low Energy communications, Wi-Fi communications, ANT communications, LoRa communications, or any combination thereof.

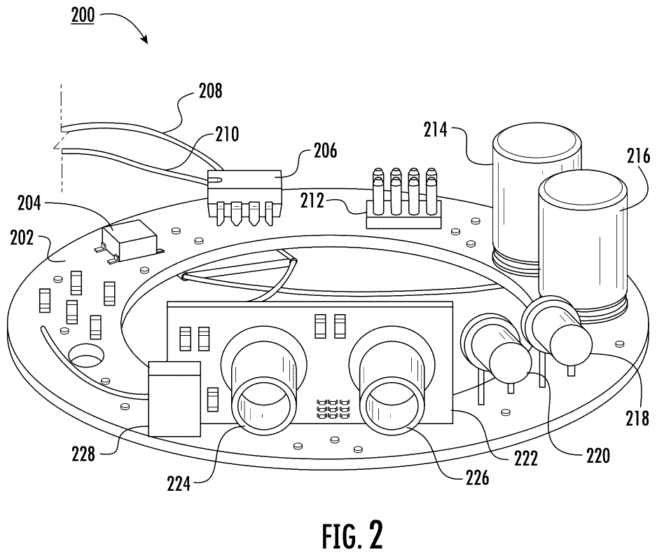

FIG. 2 shows an example of a hybrid sensor 200 in accordance with one or more aspects of the disclosure. The hybrid sensor 200 may comprise circuit board 202 that includes processor 204, memory 206, connection module 212, a first capacitor 214, a second capacitor 216, a ToF sensor 222, and/or an IR sensor comprising a first IR transmitter 218, a second IR transmitter 220, and an IR receiver 228. A data bus (not shown) may interconnect processor 204, memory 206, the first IR transmitter 218, the second IR transmitter 220, the time-of-flight sensor 222, and/or the IR receiver 228. Additionally, a first electrical lead 208 and a second electrical lead 210 may connect circuit board 202 to a power supply (not shown). The power supply may be configured to supply power to hybrid sensor 200 and/or any additional components, such as a flushing mechanism, a soap dispenser, a faucet, etc. In some instances, the power supply may be a standard alternating current (AC) connection (e.g., 120V/60 Hz). Alternatively, the power supply may be a low voltage power supply (e.g., 6 volts provided by 4 AA alkaline batteries, a lithium-ion battery, etc.) configured to power hybrid sensor 200 and/or any additional components.

Processor 204 may be any suitable processor configured to control operation of the hybrid sensor 200 and its associated components, including memory 206, the first IR transmitter 218, the second IR transmitter 220, the ToF sensor 222, and/or the IR receiver 228. Processor 204 may include a single central processing unit (CPU), which may be a single-core or multi-core processor, or may include multiple CPUs. Additionally or alternatively, processor 204 may include a low-power processor and/or microcontroller, such as an Advanced RISC Machine (ARM) processor, an Atmel 8-bit AVR microcontroller, and/or any suitable field programmable array (FPGA) or application specific integrated circuit (ASIC). Processor 204 and/or the associated components described herein may allow the hybrid sensor 200 to execute a series of computer-readable instructions to perform some or all of the processes described herein. In some examples, processor 204 may comprise an internal memory. The memory may be cache, random access memory (RAM), read only memory (ROM), electronically erasable programmable read only memory (EEPROM), flash memory, or other memory technology. The memory may be configured to store the series of computer-readable instructions that allow processor 204 to perform some or all of the processes described herein.

Memory 206 may include, but is not limited to, random access memory (RAM), read only memory (ROM), electronically erasable programmable read only memory (EEPROM), flash memory, or other memory technology, optical disk storage, magnetic cassettes, magnetic tape, magnetic disk storage or other magnetic storage devices, or any other medium that may be used to store the desired information and that may be accessed by processor 204. Software may be stored within memory 206 to provide instructions to processor 204 that allow hybrid sensor 200 to perform various actions. The various hardware memory units in memory 204 may include volatile and nonvolatile, removable and non-removable media implemented in any method or technology for storage of information such as computer-readable instructions, data structures, program modules, or other data.

Connection module 212 may be any connection interface configured to communicate with one or more control modules. For example, connection module 212 may include a plurality of pins (e.g., 4, 6, 8, 12, etc.) configured to receive a female connector from one or more control modules. In this regard, processor 204 may communicate with the one or more control modules via connection module 212. For instance, the processor 204 may send a first signal and/or power via connection module 212 to a flush control module. The flush control module may receive the first signal and provide a signal to a solenoid, which may cause a plunger to move to effectuate flushing of a toilet (or urinal). Similar operations may occur to turn on a faucet, turn off a faucet, dispense soap, activate a hand dryer, dispense paper towels, open an automatic door, etc. In another example, the processor 204 may send a second signal and/or power via connection module 212 to an ultraviolet sanitation unit (e.g., ultraviolet lights). The ultraviolet sanitation unit activate one or more ultraviolet lights. The one or more ultraviolet lights may be activated for a predetermined amount of time, for example, to neutralize, disinfect, clean, sterilize, and/or sanitize a plumbing fixture and, in particular, a surface of the plumbing fixture. Moreover, the ultraviolet light, which travels at the speed of light, may repeatedly and/or regularly irradiate individual droplets of a plume caused as a result of a flushing action, since the plume moves at a considerably slower rate than the ultraviolet light. This allows the ultraviolet light to neutralize, sterilize, sanitize, and/or kill, or otherwise mitigate, any pathogens and/or harmful material that are aerosolized as a result of the flushing action and/or dispersed via the plume, as well as those that land on any surfaces (e.g., surfaces of a water closet, surfaces of a fixture, the floor, the ceiling, etc.).

First capacitor 214 and second capacitor 216 may be capacitors of any suitable size. First capacitor 214 and second capacitor 216 may be bi-stable solenoid driver storage components. In this regard, first capacitor 214 and/or second capacitor 216 may be configured to operate a solenoid. For instance, first capacitor 214 may be configured to latch the solenoid and second capacitor 216 may be configured to unlatch the solenoid. Additionally or alternatively, first capacitor 214 and second capacitor 216 may be configured to regulate the voltage to circuit board 202, processor 204, first IR transmitter 218, second IR transmitter 220, time-of-flight sensor 222, and/or IR receiver 228.

First IR transmitter 218 and second IR transmitter 220 may be part of a proximity sensor, such as an infrared sensor. For example, first IR transmitter 218 and/or second IR transmitter 220 may be part of a Sloan® G2 proximity sensor. In some instances, first IR transmitter 218 and/or second IR transmitter 220 may be a low powered IR diode configured to emit (e.g., transmit, irradiate) IR light at a steady (e.g., constant, continuous) rate. In some examples, first IR transmitter 218 may be angled upwards, while second IR transmitter 220 may be angled downward. The first IR transmitter 218 may be angled upward between 15 and 30 degrees, and second IR transmitter 220 may be angled downward at a similar angle (e.g., between 10 and 30 degrees). By angling the first IR transmitter 218 and the second IR transmitter 220 in different directions, the hybrid sensor may better detect the presence and/or location of a user proximate to hybrid sensor 200 and its relative position with respect to other non-moving (e.g., steady-state) components within range of hybrid sensor 200. IR receiver 228 may be another component of the proximity sensor (e.g., the IR sensor). In this regard, IR receiver 228 may be a photoreceptor configured to detect IR light transmitted by the first IR transmitter 218 and/or the second IR transmitter 220. The IR receiver 228 may detect an object proximately located to hybrid sensor 200, for example, if a certain amount and/or intensity of IR light was detected. That is, if the detected light was equal to or greater than a predetermined threshold (e.g., a predetermined number of lumens), the IR receiver 228 (e.g., photoreceptor) may indicate an object proximate to hybrid sensor 200. Additionally or alternatively, several thresholds may be used to determine how close the object is to the hybrid sensor 200. Indicating an object proximate to hybrid sensor 200 may comprise sending (e.g., transmitting) a signal to processor 204 indicating the presence of the object. The first IR transmitter 218, the second IR transmitter 220, and the IR receiver 228 may be collectively referred to as an IR sensor.

ToF sensor 222 may comprise a ToF transmitter 224 and a ToF receiver 226. The ToF transmitter 224 may be a diode configured to emit (e.g. transmit, send) a laser beam at one or more objects. The ToF transmitter 224 may be a Vertical Cavity Surface-Emitting Laser (VCSEL) configured to transmit a laser at a predetermined wavelength (e.g., 940 nm). The ToF receiver 226 may be a photoreceptor configured to receive the laser beam reflected off of the one or more objects. The ToF sensor 222 may be configured to determine how far the one or more objects are from hybrid sensor 200 using the roundtrip time from when the laser was transmitted by the ToF transmitter 224 until the reflected laser was received by the ToF receiver 226. In some examples, the ToF sensor 222 may use a SPAD (Single Photon Avalanche Diodes) array to measure distances up to several (e.g., ≥2) meters away in a short period of time (e.g., <30 ms).

The hybrid sensor 200 may comprise one or more detection zones. That is, the hybrid sensor 200 may also determine how far (e.g., an IR distance) an object is from the sensor in addition to detecting the presence of the object. For example, the hybrid sensor 200 may comprise a first detection zone, a second detection zone, and/or a third detection zone. The first detection zone may be considered an entering zone, where a user makes an approach (e.g., an initial approach) toward the hybrid sensor 200. The second detection zone may be considered a using zone, where the user may be standing proximate to the hybrid sensor 200 (e.g., evacuating their bladder over a toilet, standing at a sink to wash their hands, standing at hand dryer and/or paper towel dispenser, etc.). The third detection zone may be a sitting zone, where the user may be sitting proximate to the hybrid sensor 200. It will be appreciated that the example above is merely illustrative and more, or fewer, detection zones may be employed by the hybrid sensor 200.

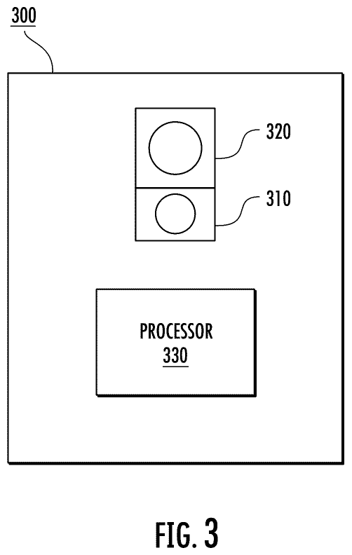

Instead of the time of flight sensor discussed above in FIG. 2, the flushometer may use an infrared (IR) sensor to activate the flushing module and/or the ultraviolet sanitation unit. FIG. 3 shows an example of an infrared sensor 300 according to one or more aspects of the disclosure. The infrared sensor 300 may be a Sloan® G2 proximity sensor and/or an APDS-9960 device from Avago Technologies. The infrared sensor 300 may comprise IR transmitter 310, IR receiver 320, and a microcontroller including a processor 330. The IR transmitter 310 may be a low powered IR diode configured to emit (e.g., transmit, irradiate) IR light at a steady (e.g., constant, continuous) rate. The IR transmitter 310 may comprise a plurality of LEDs configured to emit IR light at a particular intensity, frequency, and/or wavelength. The IR receiver 320 may comprise one or more photoreceptor cells. The one or more photoreceptor cells may be configured to detect IR light transmitted by the IR transmitter 310. Processor 330 may be any suitable processor, similar to those discussed above. The processor 330 may be configured to determine if the light detected by the IR receiver 320 is equal to or greater than a predetermined threshold (e.g., a predetermined number of lumens). If so, the processor 330 may determine that an object is proximately located near the sensor 300. The processor 330 may cause one or more signals to be sent (e.g., transmitted), which may activate a flushing module and/or an ultraviolet sanitation unit comprising one or more light arrays. If the detected light is below the threshold, the processor 330 may determine that an object is not proximate to the sensor 300. If the one or more light arrays are active (e.g., illuminated), the processor 330 may cause a signal to be sent (e.g., transmitted) to turn off the one or more light arrays. If the one or more light arrays are not active (e.g., not illuminated), then processor 330 may not send any signal.

FIGS. 4A and 4B show an example of light array assembly that is part of the ultraviolet sanitation unit. The light array assembly may comprise a socket 410 and a light array 420 formed of a plurality of light emitting devices (e.g., LED's) arranged within the socket 410. The light array 420 may comprise any suitable light array configured to generate ultraviolet light (e.g., UV-A, UV-B, UV-C). The ultraviolet light may be of a wavelength safe for human tissue. For example, the wavelength of the ultraviolet light generated by light array 420 may be 147 nm, 172 nm, 220-280 nm, or 308 nm. Preferably, the ultraviolet light generated by the light array 420 is in the range of 222 nm, which has proven effective for killing and/or destroying bacteria and viruses without being dangerous to humans and/or penetrating deeply into human cells. As noted herein, the ultraviolet light, travelling at a speed significantly greater than a plume dispersed as a result of a flushing action, may repeatedly and/or regularly irradiate individual droplets of the plume that are aerosolized or land on any surfaces, for example, via incident and/or reflected light. Moreover, the effectiveness of the ultraviolet light for cleaning, sanitizing, and/or sterilizing may be determined based on the distance travelled by the ultraviolet light and/or the rate of exposure to ultraviolet light. The socket 420 may be a 50 mm (L)×50 mm (W)×3 mm (D) square tile. This may allow for the use of multiple sockets and/or light arrays to illuminate an area. While a square tile is shown, it will be appreciated that the socket 410 and/or light array 420 may have different geometries (e.g., rectangular, hexagonal, octagonal, etc.). Socket 410 and/or light array 420 may comprise a safety mechanism to limit a user's exposure to ultraviolet light. In some instances, the light array assembly may be configured to be aware of the spread of the ultraviolet light emitted, for example, by light array 420. That is, the light array assembly may have details about the spread of ultraviolet light from the light array 420. In some embodiments, the light array assembly may be configured to adjust the spread of the ultraviolet light emitted by the light array 420. For example, the light array assembly may have a lens (not shown) to focus or spread the ultraviolet light. Preferably, the light array assembly may spread (e.g., diffuse) the ultraviolet light emitted by the light array 420 to maximize the target surface area to be cleaned, sanitized, and/or sterilized. In further embodiments, the light array assembly may have one or more LEDs capable of generating visible light (not shown). The one or more LEDs capable of generating visible light may be activated at the same time as the UV emitting LEDs. In this regard, the one or more LEDs capable of generating visible light may signal that a cleaning/sterilization/sanitation process is in progress, since the UV light is not perceptible to the human eyes. The one or more LEDs capable of generating visible light may turn off when the cleaning/sterilization/sanitation process has been completed. This may signal to a user that the cleaning/sterilization/sanitation process is over.

As noted above, the sanitation unit described herein may be activated to mitigate against the spread of pathogens and other harmful material after a fixture (e.g., toilet, urinal, etc.) has been used. FIG. 5 shows a flow chart of an example process 500 for sanitizing a plumbing fixture according to one or more aspects of the disclosure. Some or all of the steps of process 500 may be performed using one or more devices described herein.

In step 510, a sensor, such as the sensor 112, the hybrid sensor 200, or the IR sensor 300, may transmit a signal. The signal may be a laser (e.g., transmitted by the time-of-flight sensor of the hybrid sensor 200) or an IR signal (e.g., transmitted by the IR sensor 300). The signal may be transmitted constantly. Additionally, or alternatively, the signal may be transmitted intermittently. In step 520, the sensor may determine whether a user has been detected. The sensor may determine a user has not been detected if a receiver of the sensor has not detected a return signal. Accordingly, the sensor may continue to transmit a signal, as in step 510, and monitor for a user. If a receiver of the sensor has detected a return signal, the sensor may proceed to step 530 and determine whether the user is still in range of the sensor. In this regard, the time-of-flight sensor may determine that the user is still using the fixture, for example, based on a round trip time of the signal. The IR sensor may determine that the use is still using the fixture, for example, by determining if the intensity of the return IR signal satisfies a threshold. If the sensor determines that the user is still using the fixture, the sensor continues to monitor if the user is still there by returning to step 530. Additionally or alternatively, the sensor may determine which zone (e.g., first zone, second zone, and third zone discussed above) the user is in. In this regard, the sensor, or the logic of the flushometer, may determine what, if any actions, are being taken by user. A state machine may be used to track the user through each zone and/or how much time is spent in each zone. This may assist the sensor in determining when the user has left and/or finished using the fixture.

When the user has finished using the fixture, the sensor may proceed to step 540. In step 540, the sensor may send a first signal to actuate the fixture. In this regard, the sensor may send the first signal to a control unit (e.g., processor), which may activate a flush control module. Alternatively, the sensor may send the first signal directly to the flush control module. Upon receiving the first signal, the flush control module may provide a signal to a solenoid, which may cause a plunger to move to actuate the fixture. Actuating the fixture may cause a toilet or urinal to flush. Additionally, the sensor may send a second signal to actuate the sanitation unit. Like above, the second signal may be sent to a control unit or directly to the sanitation unit. In response receiving the second signal, the sanitation unit may activate one or more ultraviolet lights. The one or more ultraviolet lights may be activated for a predetermined amount of time, for example, to neutralize, disinfect, clean, sterilize, and/or sanitize a plumbing fixture (e.g., a surface of the plumbing fixture) and/or a plume dispersed by the flushing. In this regard, ultraviolet light, by virtue of travelling at the speed of light, may be well-suited to repeatedly and/or regularly irradiate individual droplets of the plume as the droplets are dispersed. The ultraviolet light may neutralize, sterilize, sanitize, and/or kill, or otherwise mitigate, any pathogens and/or harmful material that are aerosolized as a result of the flushing action and/or dispersed via the plume, as well as those that land on any surfaces (e.g., surfaces of a water closet, surfaces of a fixture, the floor, the ceiling, etc.). In some embodiments, a third signal may be sent to the sanitation unit to turn off the one or more ultraviolet lights.

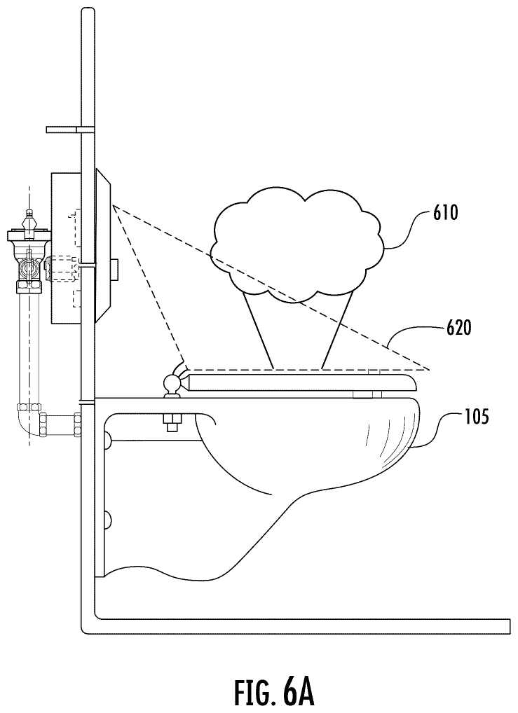

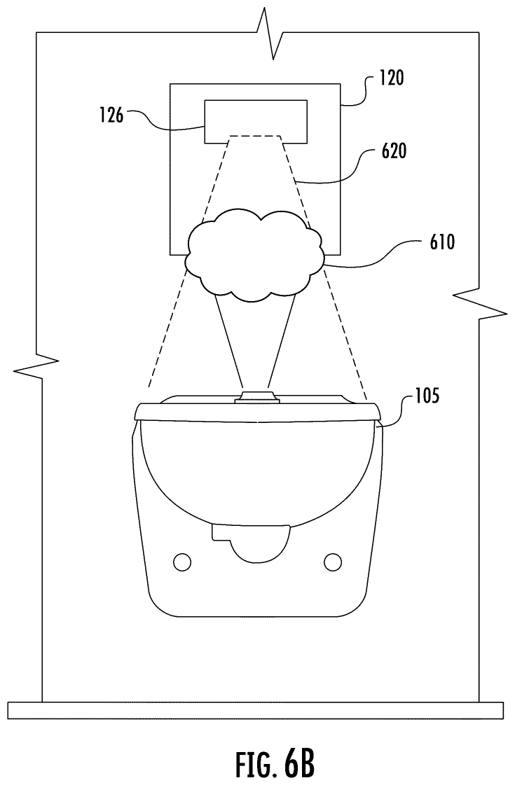

FIGS. 6A and 6B show an example of sanitizing a plumbing fixture according to one or more aspects of the disclosure. In this regard, FIGS. 6A and 6B show water closet 100, described above in FIGS. 1A and 1B. As shown in FIGS. 6A and 6B, the fixture 105 (e.g., toilet) was recently used. As a result of the recent use, the flushometer may cause the fixture 105 to be flushed, which results in plume 610 being dispersed. That is, the flushometer may cause a flushing action which causes a plume (e.g., plume 610) to be dispersed. In order to mitigate against the plume 610 dispersing pathogens and other harmful material, the sanitation unit may be activated. When activated, the sanitation unit may emit ultraviolet light 620 to kill, or otherwise mitigate, any pathogens and/or harmful material contained within plume 610. That is, the ultraviolet light 620 may travel at the speed of light, which is significantly greater than the rate of the plume's dispersal. Accordingly, ultraviolet light (e.g., ultraviolet light 620) may be well-suited to repeatedly and/or regularly irradiate individual droplets of the plume as the droplets are dispersed. This allows the ultraviolet light 620 to neutralize, sterilize, sanitize, and/or kill, or otherwise mitigate, any pathogens and/or harmful material that are aerosolized as a result of the flushing action and/or dispersed via the plume (e.g., plume 610). Moreover, the ultraviolet light 620 may kill, or otherwise mitigate, any pathogens and/or harmful material that land on the fixture 105. The effectiveness of the ultraviolet light for cleaning, sanitizing, and/or sterilizing may be determined based on the distance travelled by the ultraviolet light and/or the rate of exposure to ultraviolet light. Additionally, the sanitation unit may also emit light in a wavelength perceptible to the human eye (e.g., purple) to provide an indication that the cleaning, sterilization, and/or sanitation cycle is in progress. While a plume is shown in FIGS. 6A and 6B, it will be appreciated that the sanitation unit described herein may neutralize, sterilize, sanitize, and/or kill any pathogens contained on three-dimensional objects, as well as two-dimensional surfaces. Ideally, the ultraviolet light may be spread (e.g., diffused) to maximize the target surface area to be cleaned, sanitized, and/or sterilized. Accordingly, the sanitation unit described herein may reduce the spread of infectious diseases, while providing an environmentally friendly solution that reduces water consumption and/or further reduces waste associated cleaning chemicals and/or solutions.

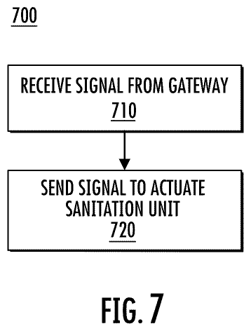

Periodically, the sanitation unit described herein may be activated as part of a cleaning, sterilization, and/or sanitation cycle. FIG. 7 shows a flow chart of an example process 700 for sanitizing a plurality of plumbing fixtures according to one or more aspects of the disclosure. Some or all of the steps of process 700 may be performed using one or more devices described herein.

In step 710, a flushometer may receive a first signal from a gateway. In this regard, a communication interface of the flushometer may receive the first signal. The first signal may be sent by a server to the flushometer, via the gateway. The first signal may indicate activation of the sanitation unit contained in the flushometer. In step 720, a second signal may be sent to the sanitation unit to activate the one or more ultraviolet lamps contained therein. The second signal may be sent from the communication interface to the sanitation unit. Alternatively, the second signal may be sent from a control unit (e.g., processor) of the flushometer after receiving the first signal via the communication interface.

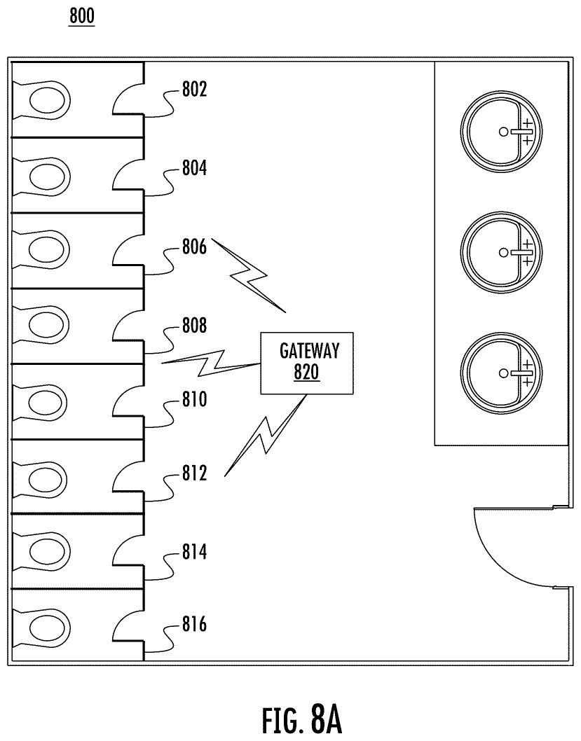

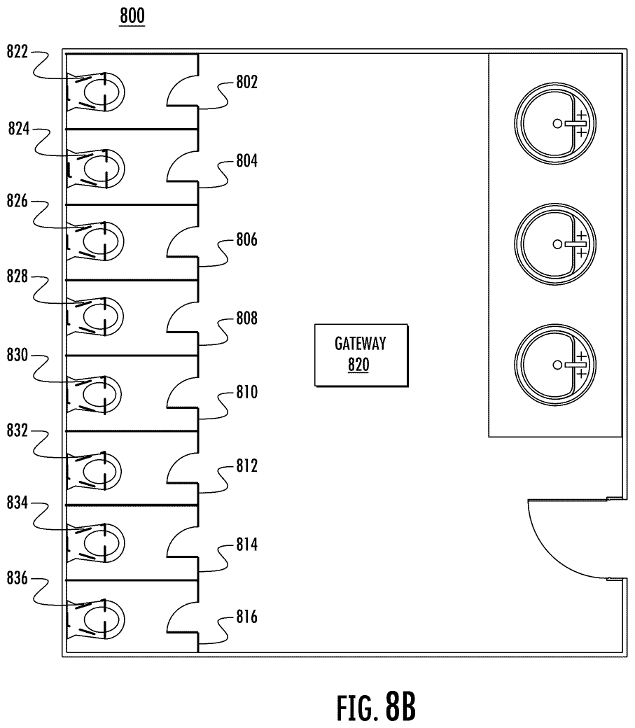

FIGS. 8A and 8B show an example of a process for sanitizing a plurality of plumbing fixtures according to one or more aspects of the disclosure. FIGS. 8A and 8B show a commercial bathroom. The commercial bathroom comprises a plurality of water closets (e.g., 802, 804, 806, 808, 810, 812, 814, and 816) and a gateway 820. The gateway 820 may receive a signal, for example, from a server indicating the initiation of a cleaning, sterilization, and/or sanitation cycle. In response to receiving the signal, the gateway 820 may transmit a signal to a sanitation unit located in each of the plurality of water closets (e.g., 802, 804, 806, 808, 810, 812, 814, and 816). The signal may be transmitted wired or wirelessly. As noted above, the sanitation unit may be located in a flushometer installed in each of the plurality of water closets (e.g., 802, 804, 806, 808, 810, 812, 814, and 816). Additionally, or alternatively, the sanitation unit may be located in the ceiling of each of the plurality of water closets (e.g., 802, 804, 806, 808, 810, 812, 814, and 816). In response to receiving the signal, each of the flushometers located in each of the plurality of water closets may activate the sanitation unit in each of the flushometers. Each of the sanitation units may cause ultraviolet light (e.g., 822, 824, 826, 828, 830, 832, 834, and 836) to be emitted therefrom. As noted above, each of the sanitation units may be angled downward to clean a surface of the toilet contained in each of the water closets (e.g., 802, 804, 806, 808, 810, 812, 814, and 816). Additionally or alternatively, the sanitation unit may comprise a motor that causes the ultraviolet light to land on the interior surface of each of the water closets. That is, the sanitation unit may move (e.g., sweep) the ultraviolet light over the interior surface of each of the water closets to disinfect, clean, and/or sanitize the surfaces of the water closets. Additionally or alternatively, the sanitation unit may cause the ultraviolet light to be swept (e.g., moved) over the surface of a plumbing fixture. If the surface to be sanitized is far from the lamp source, a longer cleaning cycle may be needed, than shorter distances. That is, the ultraviolet exposure time (e.g., how long the ultraviolet light is emitted) may be determined based on a distance the ultraviolet light has to travel and/or how long an object needs to be exposed before the object may be considered clean, sterilized, and/or sanitized. Because the effectiveness of the ultraviolet light for cleaning, sanitizing, and/or sterilizing may be based on the distance travelled by the ultraviolet light and/or the rate of exposure to ultraviolet light, the length of time for emission of the ultraviolet light may be configured, for example, based on the intended sanitation target and/or the spread of the ultraviolet light. Additionally, the sanitation unit may also emit light in a wavelength perceptible to the human eye (e.g., purple) to provide an indication that the cleaning, sterilization, and/or sanitation cycle is in progress.

It will be appreciated that the apparatuses, methods, processes, and techniques described above may sanitize surfaces without the use of any liquids, such as soap, bleach, ammonia, etc. The ultraviolet sanitation unit, and the techniques for cleaning, sterilizing, and/or sanitizing surfaces using the ultraviolet sanitation unit, described herein may reduce the spread of infectious diseases, while providing an environmentally friendly solution that reduces water consumption and/or further reduces waste associated cleaning solutions.

One or more aspects discussed herein may be embodied in computer-usable or readable data and/or computer-executable instructions, such as in one or more program modules, executed by one or more computers or other devices as described herein. Generally, program modules include routines, programs, objects, components, data structures, and the like. that perform particular tasks or implement particular abstract data types when executed by a processor in a computer or other device. The modules may be written in a source code programming language that is subsequently compiled for execution, or may be written in a scripting language such as (but not limited to) Python, Perl, or any suitable scripting language. The computer executable instructions may be stored on a computer readable medium such as a hard disk, optical disk, removable storage media, solid-state memory, RAM, and the like. As will be appreciated by one of skill in the art, the functionality of the program modules may be combined or distributed as desired in various embodiments. In addition, the functionality may be embodied in whole or in part in firmware or hardware equivalents such as integrated circuits, field programmable gate arrays (FPGA), and the like. Particular data structures may be used to more effectively implement one or more aspects discussed herein, and such data structures are contemplated within the scope of computer executable instructions and computer-usable data described herein. Various aspects discussed herein may be embodied as a method, a computing device, a system, and/or a computer program product.

Although certain specific aspects of various example embodiments have been described, many additional modifications and variations would be apparent to those skilled in the art. In particular, any of the various processes described above may be performed in alternative sequences and/or in parallel (on different computing devices) in order to achieve similar results in a manner that is more appropriate to the requirements of a specific application. Thus, embodiments disclosed should be considered in all respects as examples and not restrictive. Accordingly, the scope of the inventions herein should be determined not by the embodiments illustrated, but by the appended claims and their equivalents.

1. A system comprising:
a gateway configured to send a first signal to one or more plumbing fixtures, wherein the first signal indicates an initiation of a sanitation cycle; and
a flushometer comprising:
a wireless transceiver configured to receive the first signal; and
one or more processors configured to send a second signal to a sanitation unit in response to receiving the first signal; and
the sanitation unit configured to initiate the sanitation cycle, in response to receiving the second signal, by emitting ultraviolet light.

2. The system of claim 1, wherein the sanitation unit further comprises one or more light ultraviolet light arrays.

3. The system of claim 1, wherein the flushometer further comprises one or more light emitting diodes configure to indicate the sanitation cycle is in progress while the sanitation unit emits the ultraviolet light.

4. The system of claim 1, wherein the flushometer further comprises one or more sensors configured to send a third signal to the sanitation unit in response to detecting a user.

5. The system of claim 4, wherein the sanitation unit is configured to initiate the sanitation cycle in response to the third signal.

6. The system of claim 1, wherein the sanitation unit is configured to emit the ultraviolet light for a predetermined amount of time.

7. The system of claim 6, wherein the predetermined amount of time is determined based on a distance between the sanitation unit and an object being sanitized.

8. The system of claim 1, further comprising:
a server configured to transmit one or more signals to the gateway to indicate the initiation of the sanitation cycle.

9. The system of claim 1, wherein the flushometer comprises:
one or more sensors configured to send a third signal to the one or more processors in response to detecting a user, wherein the processors are further configured to a fourth signal to the sanitation unit that causes the sanitation unit to initiate the sanitation cycle.

10. The system of claim 9, wherein the one or more sensors comprise at least one of:
an infrared sensor;
a time-of-flight sensor; or
a hybrid sensor.

11. The system of claim 1, wherein:
the flushometer comprises a wall-mounted flushometer; and
the one or more plumbing fixtures comprise a toilet.

12. The system of claim 11, wherein the sanitation unit is configured to sweep the ultraviolet light over at least one of: an interior surface of a water closet or a surface of the toilet.

13. The system of claim 12, wherein the flushometer further comprises:
a flush control module configured to automatically flush the toilet based on the first signal, wherein the ultraviolet light is configured to neutralize pathogens in a plume created by a flushing action.

14. The system of claim 12, wherein the flushometer further comprises:
a manual flush button configured to effectuate a flushing operation of the toilet in response to being pressed, wherein the sanitation unit is configured to initiate the sanitation cycle in response to detecting the manual flush button being pressed.

15. The system of claim 1, wherein the ultraviolet light has a wavelength between 220 nanometers and 280 nanometers.

16. The system of claim 1, wherein the sanitation unit is configured to execute the sanitation cycle periodically.

17. The system of claim 1, wherein the sanitation unit further comprises:
a motor configured to move the ultraviolet light over a surface of a plumbing fixture of the one or more plumbing fixtures.

18. The system of claim 1, wherein the sanitation unit is further configured to emit light in a wavelength perceptible to a human eye to provide an indication that the sanitation cycle is in progress.

19. The system of claim 1, wherein the sanitation unit further comprises a diffuser configured to spread the ultraviolet light over a targeted surface area.

20. The system of claim 13, wherein the sanitation unit is configured to determine a time of the sanitation cycle based on a distance between the flushometer and a plumbing fixture of the one or more plumbing fixtures.
